(19) United States
(12) Patent Application Publication
(10) Pub. No.: US 2006/0058585 A1
(43) Pub. Date: Mar. 16, 2006

(54) Medical instrument set and method for creating a surgical operating space in operations on the jaw

(76) Inventors: Martin Oberlaender, Engen (DE); Stefan Rehbein, Immendingen-Hattingen (DE); Klaus M. Irion, Liptingen (DE); Michael Sauer, Tuttlingen (DE); Siegmar Reinert, Tuebingen (DE); Michael Krimmel, Tuebingen (DE); Eberhard Utz, Tuebingen (DE); Juergen Hoffmann, Tuebingen (DE)

Correspondence Address: ST. ONGE STEWARD JOHNSTON & REENS, LLC, 986 BEDFORD STREET, STAMFORD, CT, 06905-5619, US

(21) Appl. No.: 11/220,247

(22) Filed: Sep. 6, 2005

Related U.S. Application Data
(63) Continuation of application No. PCT/EP04/02139, filed on Mar. 3, 2004.

(30) Foreign Application Priority Data
Mar. 6, 2003 (DE) 103 10 978.1

(51) Int. Cl. A61B 1/32

(52) U.S. Cl. 600/210

(57) ABSTRACT

A medical instrument system for creating a surgical operating space in the human or animal body by minimally invasive way in operations on the jaw comprises a shaft, which can be introduced through an incision in the cheek, and a plate-shaped flat element which can be introduced through the mouth, a connection mechanism being provided at a distal end of the shaft and on the plate-shaped element and being designed such that the plate-shaped element can be secured on the shaft.

### CROSS REFERENCE TO RELATED APPLICATION

The present application is a continuation of pending International patent application PCT/EP2004/002139 filed on Mar. 3, 2004 which designates the United States and claims priority of German patent application 103 10 978.1 filed on Mar. 6, 2003.

### BACKGROUND OF THE INVENTION

The invention relates to a medical instrument system for creating a surgical operating space in the human or animal body by minimally invasive way in operations on the jaw.

Operations which are performed on the lower jaw, in particular in the area of the jaw joint, and which are necessary, for example, as a result of fractures of the lower jaw, have hitherto often had to be carried out by open surgery. In open surgery, extensive skin incisions are made in the patient's cheek anterior to the ear, in order to expose the lower jaw at the operating site and to be able to perform the necessary surgical measures with visual monitoring by the naked eye.

However, open surgery involves an increased potential for complications. The relatively complex approach from outside may, for example, result in injuries to the facial nerve (nervus facialis), which may cause permanent paralysis of the patient's facial muscles. In addition to these health risks, the extensive skin incisions also result in corresponding scar formations which are esthetically detrimental to the patient's appearance.

To move away from the conventional techniques of open surgery and reduce the surgical trauma, DE 197 17 977 A1 describes an instrument system with which operations on the lower jaw or operations on the jaw joint can be performed by minimally invasive surgery. With this known instrument, a bone plate for osteosynthesis can be fitted on the lower jaw in an endoscopic procedure in order to treat a fracture of the lower jaw or of the jaw joint.

The known instrument system comprises an endoscope and an endoscope supplement which receives the endoscope and also the bone plate. The endoscope supplement with the endoscope is introduced through an artificial opening made in the oral cavity on the jaw. The endoscope then permits visual monitoring through the mouth (transorally).

The known instrument system also comprises a connection tube which is introduced into the operating site from outside through the cheek via a separate incision. This connection tube is securely connected to the endoscope supplement in the area of its distal end.

The disadvantage of this known instrument system is that the bone plate is brought to the operating site via the endoscopic supplemental device, which is coupled directly to the endoscope. By this means, the necessary movements for orienting and fixing the bone plate on the jaw area to be fixed lead to an involuntary and constant changing of the viewing field, which makes visual monitoring of the procedure considerably more difficult. Moreover, securing the endoscopic fixture to the connection tube introduced from outside through the cheek requires that the distal end of the connection tube coincides with the distal part of the endoscopic supplemental device and the bone plate, which, because of the two different accesses, namely the access through the mouth and the access through the cheek, is not guaranteed.

A particular disadvantage of the known instrument system, however, is that said known instrument does not permit the creation, at the operating site, of a preformed operating space allowing work to be carried out with a clear view, which is necessary for safely performing the operation.

### SUMMARY OF THE INVENTION

It is therefore an object of the invention to make available a medical instrument system of the type mentioned at the outsystem, with which a surgical operating space can be created in the human or animal body by minimally invasive way in operations on the jaw, so that operations on the jaw joint can be performed quickly, simply and safely by minimally invasive way involving the least possible trauma.

According to an aspect of the invention, a medical instrument system for creating a surgical operating space in the human or animal body by minimally invasive way in operations on the jaw is provided, comprising a shaft having a distal end and a proximal end, which can be introduced through an incision in the cheek, a plate-shaped flat element which can be introduced through the mouth, and a connection mechanism provided at the distal end of the shaft and on the plate-shaped element and being designed such that the plate-shaped element can be secured on the shaft.

According to another aspect of the invention, a method for creating a surgical operating space by minimally invasive way in operations on the jaw is provided, comprising making a first incision through the cheek anterior to the ear, making a second incision in the mucous membrane adjacent to a site where the operation is to be carried out, introducing a plate-shaped flat element through the mouth und through the second incision between the lower jaw bone and the cheek in the area of the jaw angle, introducing an elongated shaft through the first incision towards the plate-shaped element, connecting a distal end of the shaft with the plate-shaped element, and drawing back the shaft after having being connected with the plate-shaped element to create the operating space between the lower jaw and a side of the plate-shaped element facing the lower jaw.

The instrument system according to the invention makes it possible to create a surgical operating space in the area of the lower jaw by minimally invasive way in operations on the jaw and on the jaw joint, since the shaft is introduced through a minimal incision or puncture in the cheek, and, on the distal end of the shaft lying opposite the jaw, the plate-shaped flat element is secured which has previously been introduced through the mouth to the operating site between the lower jaw and the cheek. By drawing the shaft back out of the incision, the soft tissue of the cheek can now be held away from the jaw, as a result of which, in respect of the operation to be performed, which can then be performed through the mouth, sufficient free operating space is made available for the tools used in the operation. By virtue of the plate-shaped flat configuration of the element, the creation of this operating space also takes place in an atraumatic manner since the element bears flat on the soft tissue, and injuries to the tissue are thereby avoided. In other words, the plate-shaped flat element represents a retractor element which, via the shaft, can be operated from outside the body through a minimal incision.

In a preferred embodiment, the plate-shaped element can be secured on the shaft in such a way that it extends substantially obliquely or transversely with respect to the shaft.

This measure has the advantage that the operating space can in this way be created in an especially efficient manner, because the drawing back of the shaft, in order to draw back the plate-shaped element, can take place substantially perpendicular to the cheek, which makes the handling of the instrument much easier for the physician.

In a further preferred embodiment, the plate-shaped element can be connected to the outer distal end of the shaft from the front.

This measure is of advantage in particular if endoscopic monitoring is also possible through the shaft, as will be described below, since the securing of the plate-shaped element on the shaft can then take place under visual monitoring through the shaft.

In a further preferred embodiment, the shape of the plate-shaped element is adapted to the lower jaw in the area of the jaw angle, the plate-shaped element extending approximately from the jaw base to the jaw joint.

This measure has, on the one hand, the advantage that the plate-shaped element as a whole has a large surface area, as a result of which a larger operating space can be created in the area of the jaw angle because more tissue can be held away from the jaw bone, and, on the other hand, the advantage that if, as is provided for in an embodiment to be described below, a suction and/or irrigation system is present on the plate-shaped element, it is also possible to achieve irrigation and suctioning over a large surface area at the operating site.

In a further preferred embodiment, the connection point between the plate-shaped element and the shaft is located in an edge area of the plate-shaped element, which edge area is adjacent to the jaw joint when the plate-shaped element is in the inserted state.

This measure has the advantage that the incision for introducing the shaft can be made close to the jaw angle, which on the one hand further reduces the risk of injury to the facial nerve. Moreover, this configuration has the advantage that the shaft in the area of the ear does not obstruct the operating steps performed through the mouth.

In a further preferred embodiment, the proximal end of the shaft is provided with a handle which protrudes laterally from the shaft.

The provision of a handle has the advantage that the tensile forces needed to draw back the tissue to create the operating space can be applied by the operator with sufficient manual force. The handle can in this case advantageously be connected to the shaft in such a way that the handle points toward the patient's temple in its intended use.

In a further preferred embodiment, the shaft is designed as the shaft of an endoscope.

The advantage of this is that the shaft not only satisfies the function of creating the operating space, but at the same time also the function of visual monitoring in the sense of endoscopic viewing of the operating site. In the aforementioned case, namely that the shaft is the shaft of an endoscope, the plate-shaped element can correspondingly be secured on the distal end of the endoscope.

In a preferred alternative, however, the shaft is made hollow as an insertion aid for insertion of an endoscope.

This configuration also affords the advantage that viewing is permitted via the shaft, which in particular greatly simplifies the securing of the plate-shaped element on the distal end of the shaft.

The further advantage of this measure is that the plate-shaped element does not have to be secured on the endoscope itself, so that a conventional endoscope can be used as endoscope, without structural changes having to be made to the endoscope in order to secure the plate-shaped element on the endoscope.

Yet another advantage of this design is that a relative movement is permitted between the shaft and the endoscope, for example an axial movement and also a relative rotation between the shaft and the endoscope, without the secured plate-shaped element being moved too. For example, the distal end of the endoscope in this design can also extend closer to the operating site past the distal end of the shaft and also past the plate-shaped element.

In this context, it is preferred if the plate-shaped element is transparent at least in the area of connection to the shaft.

This measure has the advantage that not only can the step of securing the plate-shaped element on the shaft be observed through the endoscope, the lower jaw itself can also be observed through the transparent area by means of the endoscope, and in particular the operating steps, for example fitting a bone plate on the jaw, can then also be viewed.

In a preferred alternative, the plate-shaped element has an opening at least in the area of connection to the shaft.

This embodiment also affords the advantage of permitting viewing not only of the plate-shaped element but also of the operating area lying behind the latter, this embodiment having the further advantage that the distal end of the endoscope can also be brought closer to the lower jaw through the plate-shaped element, while the plate-shaped element in its function as retractor plate for creating an operating space is placed further away from the lower jaw.

In a further preferred embodiment, a proximal end of the shaft is provided with an attachment piece for securing the endoscope on the shaft, the attachment piece being able to rotate about the longitudinal axis of the shaft, preferably through 360°.

This measure is of advantage in particular if the endoscope has an oblique viewing lens or side viewing lens, so that, by rotating the endoscope about its longitudinal axis, it is possible to view in all directions, without the shaft having to be rotated, with the result that the plate-shaped element, in accordance with its function of creating an operating space, does not have to change position in contact with the soft tissue to be held away.

In preferred embodiments of the connection mechanism for connecting the plate-shaped element to the shaft, the connection mechanism has a catch mechanism and/or a screw mechanism.

These kinds of connection mechanisms have the advantage of rapid and simple handling while at the same time ensuring reliable securing of the plate-shaped element on the shaft, the connection mechanisms preferably being generally designed such that they can be actuated from outside the body, without awkward maneuvers having to be performed in the operating site.

In a first preferred specific embodiment of the connection mechanism, the shaft has, at the distal end, a plug which extends substantially transversely with respect to the longitudinal direction of the shaft, the plate-shaped element having a substantially complementary opening, and the plug being able to be fitted into the opening in a first position of rotation and, by rotating it to a second position of rotation, being secured against withdrawal from the opening.

In this “keyhole” configuration, the plate-shaped element can be locked onto the shaft with positive engagement inside the body by means of a simple plug-in and rotational movement, the positive fit avoiding undesired detachment of the plate-shaped element from the shaft at the necessary tensile load.

In this case, it is preferred if the shaft has an inner shaft, on which the plug is formed, and an outer shaft which is axially displaceable but not rotatable with respect to the inner shaft, and which has, at a distal end, at least one projection which is offsystem by angle with respect to the plug about the longitudinal direction and which, with the plug in the second position of rotation, engages in the opening.

It is of advantage here that the outer shaft secures the positive connection between the plug of the inner shaft and the opening of the plate-shaped element such that the shaft as a whole is connected to the plate-shaped element in a manner fixed against rotation, and the outer shaft ensures that, if a relative torque is exerted between the shaft and the plate-shaped element, the plug does not move back unwanted into the first position of rotation. Only when the outer shaft is drawn back axially can the inner shaft with the plug be moved back from the second position of rotation to the first position of rotation in order to be able to detach the plate-shaped element from the shaft.

It is also preferred if the outer shaft is pretensioned in the distal direction by means of a spring force.

It is of advantage here that, upon connection of the plate-shaped element to the shaft, the outer shaft in the second position of rotation snaps automatically into the opening, as a result of which the handling of the instrument according to the invention when securing the plate-shaped element on the shaft is further simplified.

In a second preferred structural embodiment of the connection mechanism, the shaft has an inner shaft which has a fork at its distal end, the plate-shaped element having at least two thread segments, and the fork being able to be coupled to the thread segments in a cross-over arrangement secure against rotating, and the shaft moreover having an outer shaft whose distal end can be screwed onto the thread segments.

In this embodiment too, the shaft is designed in two parts, the inner shaft according to one embodiment securing against rotation between the plate-shaped element and the shaft, while the outer shaft secures the plate-shaped element on the shaft in the axial direction.

It will be appreciated, however, that the shaft can also advantageously be designed in one piece for the purposes of a simple construction and can simply be screwed onto the plate-shaped element.

In a third preferred structural embodiment, the shaft has an inner shaft and an outer shaft, the outer shaft being able to be screwed onto the plate-shaped element, and the inner shaft being able to be secured on the outer shaft by means of a securing element and being able to be withdrawn from the outer shaft by loosening of the securing element.

In contrast to the embodiments described above, here only the outer shaft is secured on the plate-shaped element, whereas the inner shaft is held in a fixed position with respect to the plate-shaped element indirectly via the securing element and the outer shaft.

In a further preferred embodiment, the plate-shaped element has at least one suction and/or irrigation line with at least one suction and/or irrigation opening.

In this embodiment, the plate-shaped element advantageously also has the function of a suction and/or irrigation instrument, so that it is possible to dispense with introducing a separate suction and/or irrigation instrument into the operating site, with the result that the operating space receives at any one time the smallest possible number of different instruments that could obstruct the operating procedure. By suctioning and irrigation, the visual monitoring in particular through the above-described endoscope can be maintained by means of clear irrigation fluid being flushed through and by tissue and blood residues being suctioned off.

In a further preferred embodiment, the shaft has at least one suction and/or irrigation channel.

It is of advantage here that the shaft also acquires a further function, namely that of delivering irrigation fluid and/or suctioning off liquids, blood and tissue parts from the operating site.

Irrigation is preferably performed through the cheek via the shaft, whereas material is suctioned off through the mouth via the plate-shaped element.

In connection with one of the aforementioned embodiments according to which the shaft comprises an inner shaft and an outer shaft, the inner shaft being able to be secured on the outer shaft by means of a securing element, provision is made, in a further preferred embodiment, for the securing element to be a suction and/or irrigation attachment piece for attachment of a suction and/or irrigation line.

It is of advantage here that it is possible to dispense with a separate securing element for securing the inner shaft on the outer shaft, because the suction and/or irrigation attachment piece takes over this function, thus keeping down the structural outlay of the shaft.

In a further preferred embodiment, the plate-shaped element has at least one position sensor for detecting the position and/or the area of connection of the plate-shaped element to the shaft.

The at least one position sensor can be used, instead of or in addition to visual monitoring, to observe the position of the plate-shaped element in the operating site and/or to make it easier to secure the plate-shaped element on the shaft.

In this case it is preferred if the at least one position sensor is an electromagnetic sensor.

An electromagnetic sensor has the advantage that, in contrast to light, the signal transmission can also take place unimpeded by tissue.

In a further preferred embodiment, the plate-shaped element has at least one light-emitting element for radiating light through the cheek.

This embodiment too has the advantage that detection can be effected through the cheek by radiation of light from the plate-shaped element.

Further advantages and features are system out in the description below and in the attached drawing.

It will be appreciated that the features mentioned above and the features still to be explained below can be used not only in the respectively cited combination, but also in other combinations or singly, without departing from the scope of the present invention.

### BRIEF DESCRIPTION OF THE DRAWINGS

Illustrative embodiments of the invention are shown in the drawing and are described in more detail below with reference to said drawing, in which:

FIG. 1 shows a schematic perspective representation of an instrument system for creating a surgical operating space in operations on the jaw, the instrument system being shown in a simplified representation in its intended use;

FIG. 2 shows a side view of a shaft of an instrument system for the use represented in FIG. 1, according to another illustrative embodiment;

FIG. 3 shows a side view of a plate-shaped element for use with the shaft from FIG. 2;

FIG. 4 shows the plate-shaped element from FIG. 3 in a perspective representation in which it has been turned through approximately 180° in relation to FIG. 3;

FIG. 5 shows an exploded perspective representation of the whole instrument according to FIGS. 2 to 4;

FIGS. 6a)-6d) show perspective partial views of the connection mechanism between the plate-shaped element according to FIGS. 3 and 4 and the shaft according to FIG. 2;

FIG. 7 shows a perspective representation, on a smaller scale, of the instrument according to FIG. 5 during securing of the plate-shaped element on the shaft;

FIG. 8 shows a representation of the instrument corresponding to FIG. 7, the plate-shaped element now secured on the shaft;

FIGS. 9a) and 9b) show representations of the connection mechanism for connecting the plate-shaped element to the shaft, partially in longitudinal section, FIG. 9a) corresponding to the representation in FIG. 6b), and FIG. 9b) corresponding to the representation in FIG. 6d);

FIG. 10 shows a perspective exploded representation of an instrument system according to another illustrative embodiment;

FIG. 11 shows a side view of the shaft of the instrument system from FIG. 10 in isolation;

FIG. 12 shows a detailed representation of the connection mechanism for connecting the plate-shaped element of the instrument system from FIG. 10 to the associated shaft according to FIG. 10 and FIG. 11, on a larger scale, FIG. 12 showing the connection mechanism before the plate-shaped element is secured on the shaft;

FIG. 13 shows a representation corresponding to FIG. 12, and partially in longitudinal section, the plate-shaped element now secured on the shaft;

FIG. 14 shows a perspective representation of an instrument system according to a further illustrative embodiment, the plate-shaped element of the instrument system being shown separated from the shaft;

FIG. 15 shows a perspective exploded representation of the shaft of the instrument system from FIG. 14 without the plate-shaped element;

FIG. 16 shows a distal end of the shaft of the instrument system from FIG. 14 and FIG. 15 in longitudinal section and on a larger scale, a cap being fitted on the distal end of the shaft; and

FIG. 17 shows a perspective representation of the cap from FIG. 16 in isolation.

### DETAILED DESCRIPTION OF PREFERRED EMBODIMENTS

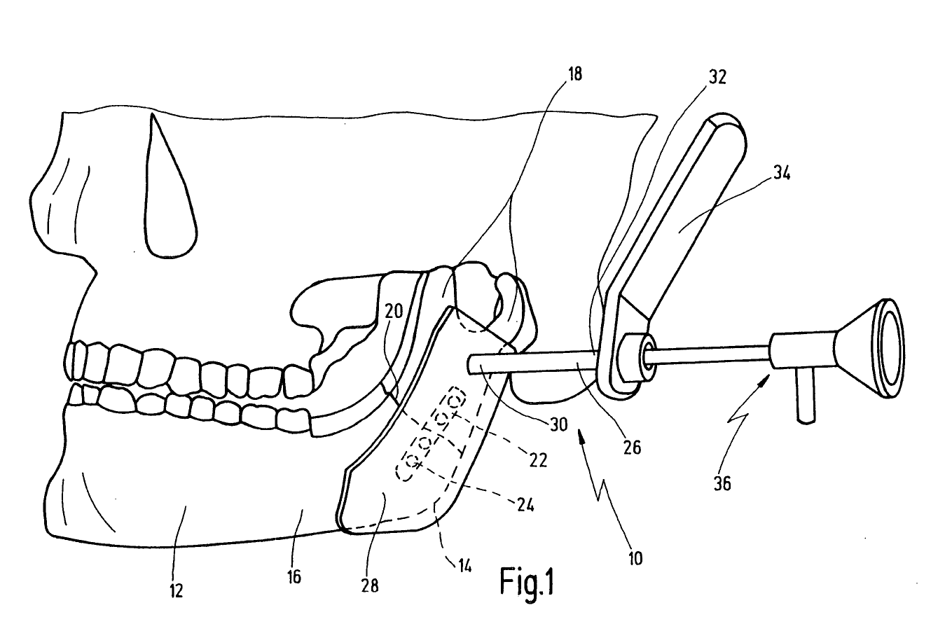

In FIG. 1, general reference number 10 designates a medical instrument system for creating a surgical operating space in the human or animal body by minimally invasive means in operations on the jaw. The instrument system 10 is used in particular in operations on the jaw joint.

FIG. 1 also shows a lower jaw 12 of a patient, the jaw angle being indicated by 14, the jaw base by 16, and the jaw joint by 18. An operation on the jaw joint may be necessary, for example, as a result of a fracture 20 of the lower jaw in the area between the jaw angle 14 and the jaw joint 18. In this operation, the fracture 20 is treated by means of a bone plate 22 which, spanning the fracture 20, is screwed onto the lower jaw 12 by means of screws 24.

The instrument 10 generally comprises a shaft 26 and a plate-shaped flat element 28. The plate-shaped flat element 28 can be secured on a distal end 30 of the shaft 26 by means of a connection mechanism to be described below, specifically after the element 28 has been introduced through the mouth and the shaft 26 has been introduced through the cheek, the element 28 being able to be secured on the shaft 26 from outside the body.

The plate-shaped element 28 is secured on the shaft 26 such that it extends substantially obliquely or transversely with respect to the latter, as is shown in FIG. 1.

In terms of its shape, the plate-shaped element 28 is adapted to the lower jaw 12 in the area of the jaw angle 14 and extends approximately from the jaw base 16 to the jaw joint 18. Adapted “to the shape of the lower jaw 12” means here that the plate-shaped element 28 is slightly curved in accordance with the natural curvature of the surface of the lower jaw 12 in the area from the jaw base 16 to the jaw joint 18.

As will further be seen from FIG. 1, the connection point between the plate-shaped element 28 and the shaft 26 is located close to that edge of the plate-shaped element 28 adjacent to the jaw angle 28.

In an operation on the jaw joint, the shaft 26 is introduced through the cheek via a small incision anterior to the ear. In the oral cavity, an incision is made in the mucous membrane adjacent to the operating site. The plate-shaped element 28 is then introduced through the mouth and via the incision in the mucous membrane between the lower jaw bone 12 in the area of the jaw angle 14 and the (muscle) tissue of the cheek. The plate-shaped element 28 is then connected from the front to the distal tip of the shaft 26, as will be described later. By drawing back the shaft 26 from the incision in the cheek, the plate-shaped element 28 is then drawn back against the outerlying tissue, as a result of which an operating space between the lower jaw 12 and that side of the plate-shaped element 28 facing the lower jaw 12 is created in which the bone plate 22 can then be securely and quickly fitted with suitable tools.

To make it easier to draw the plate-shaped element 28 back by means of the shaft 26, a proximal end 32 of the shaft 26 is provided with a handle 34 which protrudes laterally from the shaft 26 and has approximately at least the length of the width of a hand. When the instrument system 10 is in the state of use, the handle 34 points approximately in the direction of the patient's temple.

The shaft 26 of the instrument system 10 is also used for visual monitoring of the operating site. While for this purpose the shaft 26 can itself be designed as the shaft of an endoscope, for example with an endoscope optical system arranged in the shaft 26 or with the shaft 26 possibly designed as the shaft of a video endoscope, the shaft 26 in the present illustrative embodiment is hollow and thus serves as an insertion aid for insertion of an endoscope 36.

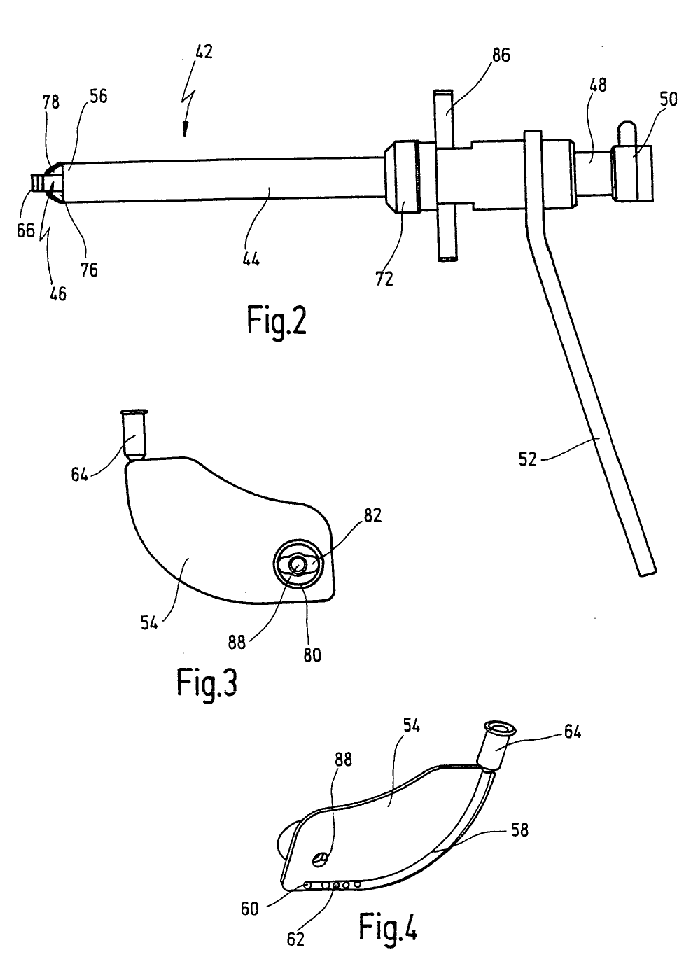

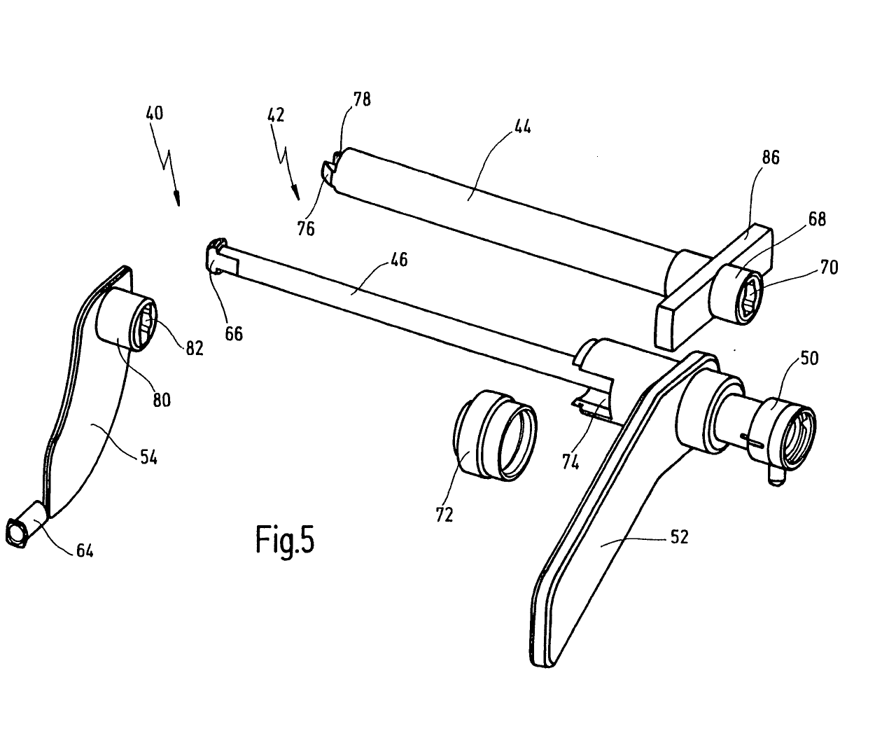

By comparison with the outline representation of the instrument system 10, FIGS. 2 to 9 show a detailed illustrative embodiment of an instrument system 40 (cf. FIG. 5) which is described below and which can be used instead of the instrument system 10 and in the same way as is shown in FIG. 1.

The instrument system 40 has a shaft 42 which corresponds to the shaft 26 of the instrument system 10 and which is formed by an outer shaft 44 and an inner shaft 46. The outer shaft 44 can be detached from the inner shaft 46 according to FIG. 5. Like the shaft 26, the shaft 42 serves as an insertion aid, for example for the endoscope 36 in FIG. 1, and at a proximal end 48 it has an attachment piece 50 through which the endoscope 36 can be inserted into the shaft 42, and by means of which the endoscope 36 can be secured on the shaft 42. The attachment piece 50 can rotate through 360° about the longitudinal axis of the shaft 32, such that the endoscope 36 together with the attachment piece 50 can be rotated about the longitudinal axis of the shaft 42 without the shaft 42 as a whole having to be rotated.

The attachment piece 50 is in this case arranged on the inner shaft 46, i.e. the inner shaft 46 receives the endoscope 36 and is correspondingly hollow throughout.

Arranged at the proximal end 48 of the shaft 42 there is also a handle 52 corresponding to the handle 34 of the instrument system 10 in FIG. 1 and secured, according to FIG. 5, on the inner shaft 46.

The instrument system 40 also has a plate-shaped flat element 54 which corresponds to the plate-shaped element 28 of the instrument system 10 in FIG. 12 and which can be secured on a distal end 56 of the shaft 42 via a connection mechanism still to be described below.

The plate-shaped element 54 has a suction line 58 arranged on one side of the plate-shaped element 54 which, in the intended use of the instrument system 40 according to FIG. 1, is directed toward the lower jaw 12. The suction line 58, which extends along a lower edge of the plate-shaped element 54, has at its end a suction opening 60 and, if appropriate, on its outside, further suction openings 62. At its end directed toward the mouth during its intended use, the suction line 58 has an attachment piece 64 for attachment of a suction hose (not shown). However, the suction line 58 can also be used as an irrigation line, if so desired.

Starting from FIG. 5, the assembly of the shaft 42 and the securing of the plate-shaped element 54 on the shaft 42 will now be described.

At the distal end 56 of the shaft 42, more precisely at the distal end of the inner shaft 46, there is a plug 68 which extends transversely with respect to the inner shaft 46 and, with the latter, forms approximately the shape of a T. At its proximal end, the outer shaft 44 has a sleeve-like projection 68 which has an opening 70 substantially complementing the plug 66. Before the start of the operation, the outer shaft 44 is pushed onto the inner shaft 46 from the distal end thereof, and, because of the configuration of the opening 70, the outer shaft 44 can be pushed onto the inner shaft 46, via the plug 66, only in a defined rotation position relative to the inner shaft 46. A sleeve nut 72 is then pushed onto the outer shaft 44 and, according to FIG. 2, is screwed together with the inner shaft 46. The outer shaft 44 is then connected in a rotationally fixed manner to the inner shaft 46, but can still be displaced axially relative to the inner shaft 46. The outer shaft 44 is pretensioned in its maximum distal position shown in FIG. 2 by means of a spring force, for example by a spring (not shown) arranged in a sleeve-like projection 74 of the inner shaft 46, and it can be drawn back in the proximal direction counter to the spring force.

At its distal end, the outer shaft 44 has two projections 76, 78 which are offsystem by 180° relative to one another and which, when the outer shaft 44 is mounted on the inner shaft 46, are offsystem by an angle to the plug 66 of the inner shaft 46, specifically by 90°.

After the preliminary assembly of the shaft 42 as described above, the shaft 42 can now be introduced through the cheek via a small incision anterior to the ear, as has been described in connection with FIG. 1.

The plate-shaped element 54, which is introduced through the mouth and through the incision in the mucous membrane as described in connection with FIG. 1, has a sleeve-like projection 80 having an opening 82 which is of a substantially complementary design to the plug 66.

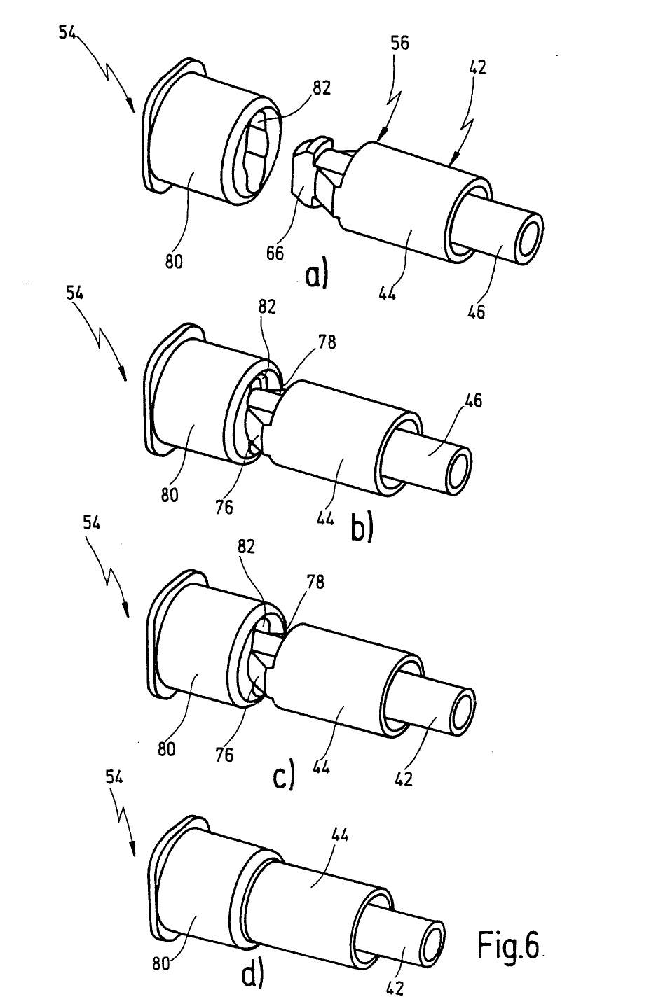

The securing of the plate-shaped element 54 on the shaft 42 is now described with reference to FIGS. 6 through 9.

In FIGS. 6a) to 6d), the plate-shaped element 54 is shown only in the area of the projection 80, and the shaft 42 is shown only in the area of its distal end 56. The same applies to FIGS. 9a) and 9b).

Starting from FIG. 6a), the shaft 42 with the plug 66 to the front is introduced into the opening 82 of the projection 80 in the manner of a key, and, because of the configuration of the plug 66 and the substantially complementary configuration of the opening 82, this is possible only in a defined position of rotation of the shaft 42 relative to the plate-shaped element 54. This state is shown in FIG. 6b) and FIG. 9a). The projections 76 and 78 of the outer shaft 44 come to bear on the edge of the opening 82, as is shown in FIG. 6b) and FIG. 9a).

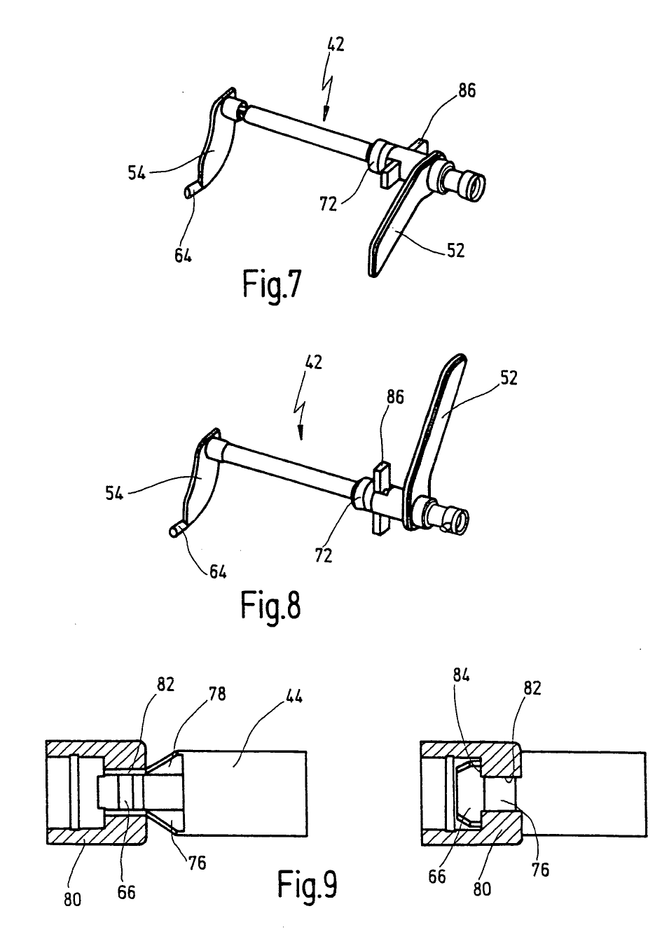

By pushing the shaft 42 farther forward according to FIG. 6c), the plug 66 engages completely in the opening 82, while the outer shaft 44 is pushed back relative to the inner shaft 46. This position is shown not only in FIG. 6c) but also in FIG. 7. Starting from this position, the shaft 42 is now turned via the handle 52 into a second position of rotation about the longitudinal axis of the shaft 42 (see FIG. 8), such that the plug 66, bearing with a positive fit on an undercut 84 of the opening 82, is secured against withdrawal. The outer shaft 44 connected to the inner shaft 46 in a rotationally fixed manner is likewise turned through 90°, so that the projections 76 and 78 then snap into the opening 82, as a result of which the shaft 42 as a whole is connected to the plate-shaped element 54 fixed against rotation and against pulling. This state is shown in FIG. 6d), FIG. 8 and FIG. 9b).

To release the plate-shaped element 54 from the shaft 42 on completion of the operation, the reverse procedure is carried out. First, the outer shaft 44 is drawn back relative to the inner shaft 46 counter to the spring force, for which purpose a grip element 86 is arranged at the proximal end of the outer shaft 44. After the outer shaft 44 has been drawn back, the entire shaft 42 can then be turned through 90° via the handle 52, after which the shaft 42 can be removed from the plate-shaped element 54.

Before the plate-shaped element 54 is secured on the shaft 42, the endoscope 36 in FIG. 1 can be inserted into the shaft 42 so that the securing process can be monitored through the endoscope 36. Also provided in the plate-shaped element 54 there is an opening 88 which is flush with the opening 82 and through which the individual operating steps can be visually monitored via the endoscope 36 after the plate-shaped element 54 has been secured on the shaft 42. If practicable, the endoscope 36 can also be inserted with its distal end through the opening 88 so that the endoscope can be system at different distances from the lower jaw 12.

As an alternative to the opening 88, or in addition to the opening 88, the plate-shaped element 54 can also be made transparent at least in the area of connection to the shaft 44.

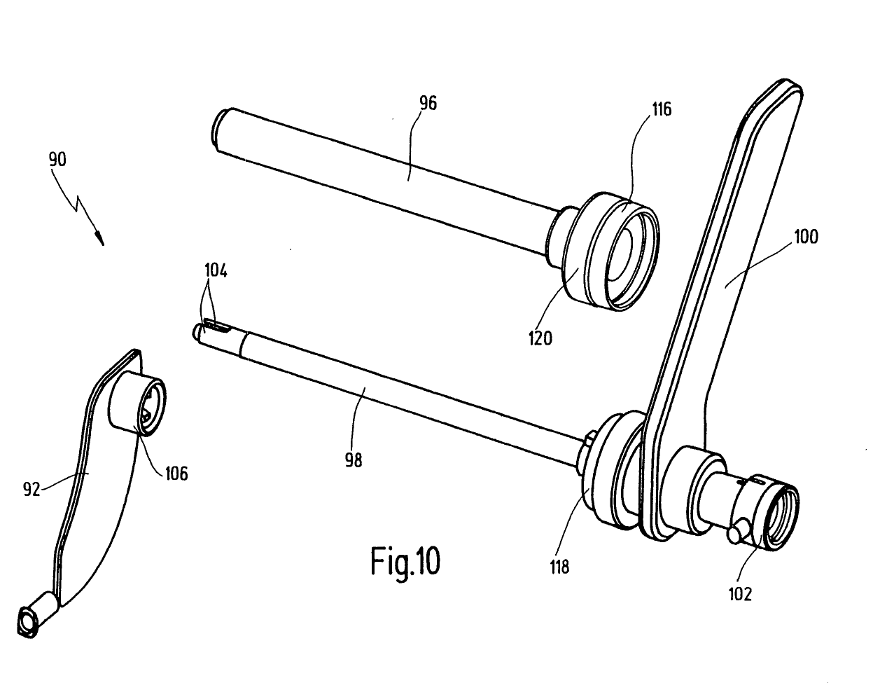

While the connection mechanism described above in relation to FIGS. 2 to 9 is a catch mechanism in which the shaft 42 can be locked to the plate-shaped element 54, a further illustrative embodiment of an instrument system 90 with reference to FIGS. 10 to 13 is described in which the connection mechanism for securing a plate-shaped element 92 on a shaft 94 is based principally on a screw mechanism.

Unless otherwise indicated below with reference to the instrument system 90, the configuration of the instrument system 90 otherwise corresponds to the instrument system 10 or instrument system 30.

As in the previous illustrative embodiment, the shaft 94 comprises an outer shaft 96 and an inner shaft 98, and also a handle 100 which is connected to the inner shaft 98, and an attachment piece 102 for insertion and securing of the endoscope 36 from FIG. 1 for example, the attachment piece 102 likewise being arranged on the inner shaft 98.

At its distal end, the inner shaft 98 has a fork 104, i.e. the inner shaft 98 has a slotted configuration at its distal end. The plate-shaped element 92 has a sleeve-shaped projection 106 in whose interior two thread segments 108 and 110 are arranged offsystem from one another by 180°. The thread segments 108 and 110 have a thread on their outside 112, as is shown for the thread segment 110 in FIG. 13.

At its distal end, the outer shaft 96 has an internal thread 114.

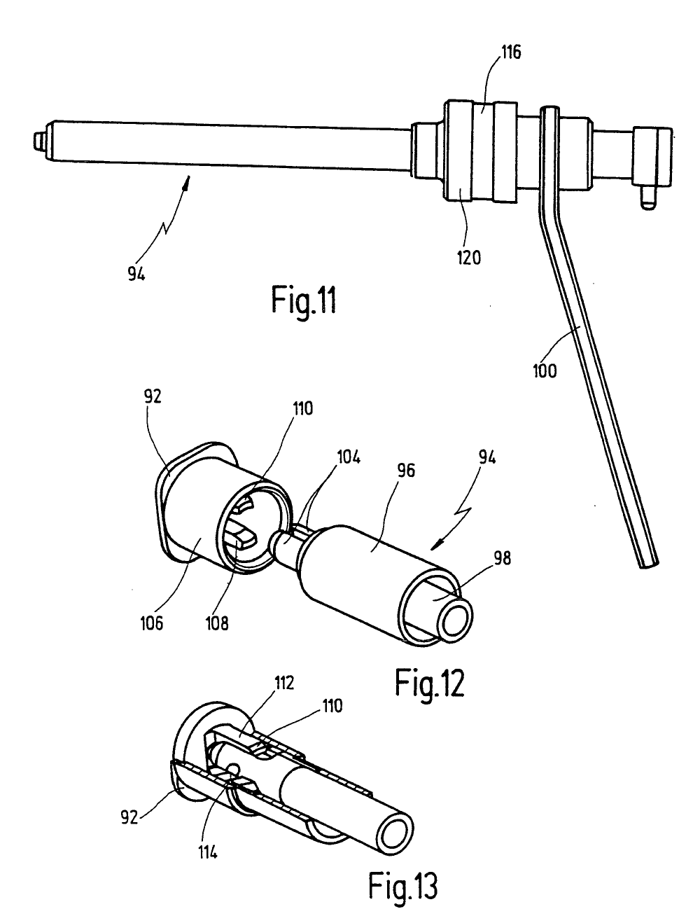

Starting from FIG. 10, the outer shaft 96 is first pushed onto the inner shaft 98 so that the shaft 94 is assembled as shown in FIG. 11. Here, the outer shaft 96 is secured on the inner shaft 98 via a sleeve nut 116 which can be screwed onto a threaded piece 118 on the inner shaft 98. However, the outer shaft 96 remains free in rotation relative to the inner shaft 98 as the sleeve nut 116 is adjoined by a swivel joint 120. To connect the thus pre-assembled shaft 94 to the plate-shaped element 92, the shaft 94 with the fork 104 at the front is pushed into the projection 106 of the plate-shaped element 92, the fork being coupled to the thread segments 108 and 110, as shown in FIG. 13, in an arrangement crossed over by 90°. The inner shaft 96 is then connected to the plate-shaped element 92 in a rotationally fixed manner. Then, according to FIG. 13, the outer shaft 96 is screwed with its internal thread 114 onto the thread segments 108 and 110, so that the shaft 94 is then also secured in the axial direction on the plate-shaped element 92. The release of the shaft 94 from the plate-shaped element 94 thus takes place in the reverse sequence.

The handle 100 is oriented with respect to the fork 104 in such a way that, when the plate-shaped element 92 is secured on the shaft 94, the handle 100 is pointing toward the patient's temple when the instrument system 90 is in its intended use according to FIG. 1.

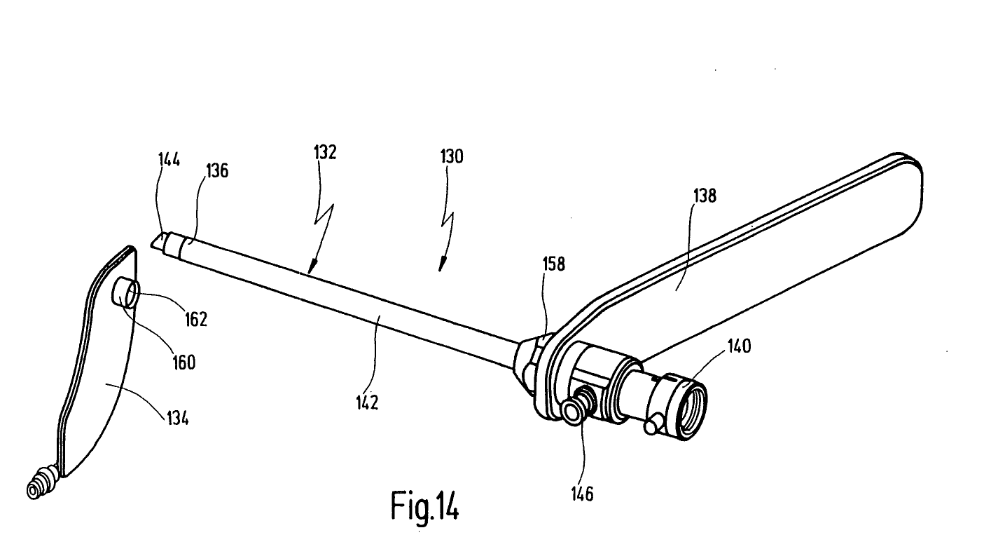

Referring to FIGS. 14 to 17, a further illustrative embodiment of an instrument system 130 is described which can be used similarly to the instrument system 10 according to FIG. 1.

Unless otherwise stated below, features of the instrument system 130 which are not described below are identical to the features of the instrument systems 10, 40 and 90 or at least correspond to these features.

The instrument system 130 has a shaft 132 and a plate-shaped element 134 which can be secured on a distal end 136 of the shaft 132 by means of a connection mechanism still to be described below.

At its proximal end, the shaft 132 has a handle 138 protruding laterally from the shaft 132. At the outermost proximal end, the shaft 132 has an attachment piece 140 for securing for example the endoscope 36 from FIG. 1 on the shaft 132, the endoscope 36 being able to be inserted into the shaft 132 as has been described already with reference to the previous illustrative embodiments.

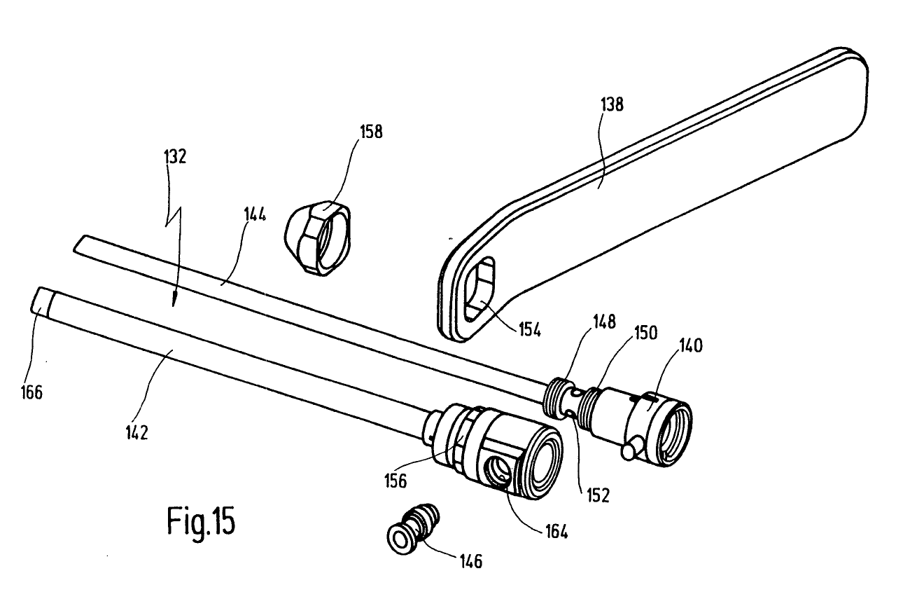

The shaft 132 comprises an outer shaft 142 and an inner shaft 144. The outer shaft 142 can be pushed onto the inner shaft 144, as is shown in FIG. 14. In FIG. 15, the outer shaft 142 and the inner shaft 144 are shown in the state when detached from one another.

The inner shaft 144, which has the attachment piece 140 at its proximal end, serves not only as an insertion aid for an endoscope, for example the endoscope 36 in FIG. 1, but is at the same time designed as an irrigation shaft and has a corresponding irrigation channel (not shown in detail).

Arranged at the proximal end of the shaft 132 there is accordingly an irrigation attachment piece 146 which, in addition to the function of an attachment piece for an irrigation line (not shown), has a further function, namely that of securing the inner shaft 144 on the outer shaft 142, as will be described below. Seals 148 and 150 seal off the inner shaft 144 from the outer shaft 142. Between the seals 148 and 150, the inner shaft 144 has openings 152, into one of which the attachment piece 146 can be screwed, in which case the corresponding opening 152 then serves as an inlet opening for irrigation fluid into the inner shaft 144.

The handle 138 can be detached from the shaft 132. The handle 138 is pushed onto the outer shaft 142 by way of an opening 152. The opening 154 is substantially rectangular, and a complementary square 156 is provided on the outer shaft 142, and the opening 154 can be fitted with positive locking onto the square 156 in four different positions of rotation. To secure the handle 138 on the outer shaft 142, a sleeve nut 158 is provided.

The plate-shaped element 134 has a sleeve-like projection 160 having a substantially cylindrical opening 162.

Before the operation, the shaft 132 is assembled by pushing the inner shaft 144 into the outer shaft 142. The attachment piece 146 is then screwed through an opening 164 in the proximal end of the outer shaft 142 and into one of the openings 152, as a result of which the inner shaft 144 is secured on the outer shaft 142 in a manner in which it is fixed in terms of rotation and is also axially non-displaceable. The handle 138 is pushed onto the outer shaft 142 and secured by means of the sleeve nut 158.

As has been described with reference to FIG. 1, the shaft 132 pre-assembled in this way can be introduced into the operating site through the incision anterior to the ear.

At its distal end, the outer shaft 142 has an external thread 166, while the opening 162 of the projection 160 of the plate-shaped element 134 has a corresponding internal thread.

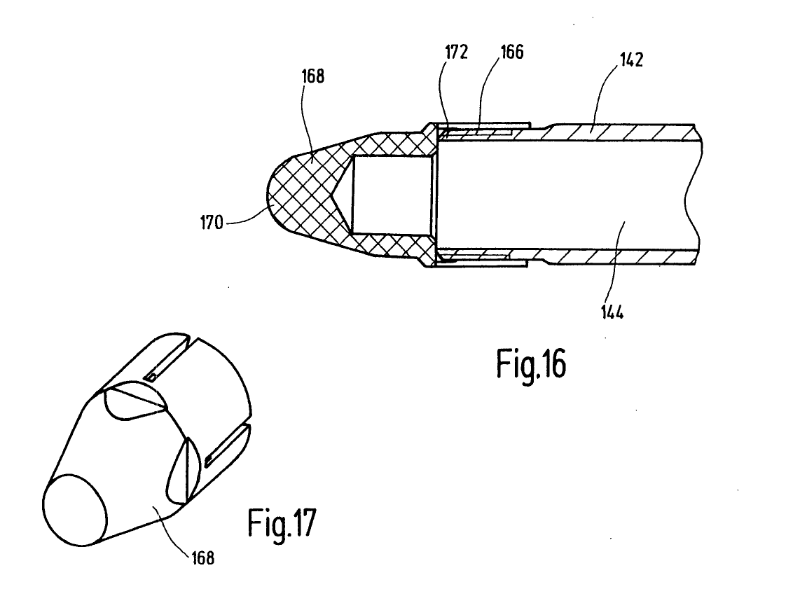

Before the shaft 132 is introduced into the incision, a cap 168 according to FIG. 17 is screwed onto the external thread 166 of the outer shaft 142, as is shown in FIG. 16, in order to make it easier to introduce the shaft 132 into the incision. The cap 168 is provided with a rounded end 170 and covers the external thread 166 and the tip 172 of the shaft 132, so that the cap 168 makes introduction of the shaft 132 into the incision atraumatic.

The cap 168 can then be unscrewed from the direction of the inside of the oral cavity, after which the shaft 132, or more precisely the outer shaft 142, can then be screwed into the thread of the opening 162 of the projection 160 of the plate-shaped element 134 which has been introduced beforehand through the mouth, after which the plate-shaped element 134 is secured on the shaft 132.

As has already been described above, the handle 138 can be brought to a desired position by appropriate loosening of the sleeve nut 158, preferably in such a way that the handle 138, as shown in FIG. 1 with reference to the instrument 10, is directed toward the patient's temple in the intended use of the instrument 130.

It will be appreciated that the illustrative embodiments cited above can also be combined with one another in terms of their structural configuration. In other embodiments not shown, the respective plate-shaped element 28, 54, 92 or 134 can further be provided with at least one position sensor for detecting the position and/or the area of connection of the respective plate-shaped element to the respective shaft 26, 42, 94 or 132. Such a position sensor is preferably an electromagnetic sensor.

It is also possible to provide, on the aforementioned plate-shaped elements, at least one light-emitting element for radiating light through the patient's cheek.

What is claimed is:

1. A medical instrument system for creating a surgical operating space in the human or animal body by minimally invasive way in operations on the jaw, comprising:
a shaft having a distal end and a proximal end, which can be introduced through an incision in the cheek,
a plate-shaped flat element which can be introduced through the mouth, and
a connection mechanism provided at said distal end of said shaft and on said plate-shaped element and being designed such that said plate-shaped element can be secured on said shaft.

2. The instrument system of claim 1, wherein said plate-shaped element can be secured on said shaft in such a way that said plate-shaped element extends at least approximately transversely with respect to said shaft.

3. The instrument system of claim 1, wherein said plate-shaped element can be connected to said distal end of said shaft from the front.

4. The instrument system of claim 1, wherein a shape of said plate-shaped element is adapted to the lower jaw in the area of the jaw angle and extends approximately from the jaw base to the jaw joint.

5. The instrument system of claim 1, wherein a point of connection between said plate-shaped element and said shaft is located in an edge area of said plate-shaped element, which edge area is adjacent to the jaw joint when said plate-shaped element is in the inserted state.

6. The instrument system of claim 1, wherein said proximal end of said shaft is provided with a handle which protrudes laterally from said shaft.

7. The instrument system of claim 1, wherein said shaft is designed as a shaft of an endoscope.

8. The instrument system of claim 1, wherein said shaft is designed as an insertion aid for insertion of an endoscope.

9. The instrument system of claim 1, wherein said plate-shaped element is transparent at least in an area of connection to said shaft.

10. The instrument system of claim 1, wherein said plate-shaped element has an opening at least in an area of connection to said shaft.

11. The instrument system of claim 1, wherein said proximal end of said shaft is provided with an attachment piece for securing an endoscope on said shaft, said attachment piece being able to rotate about a longitudinal axis of said shaft.

12. The instrument system of claim 1, wherein said connection mechanism for connecting said plate-shaped element to said shaft has a catch mechanism.

13. The instrument system of claim 1, wherein said connection mechanism for connecting said plate-shaped element to said shaft has a screw mechanism.

14. The instrument system of claim 1, wherein said connection mechanism comprises a plug arranged at said distal end of said shaft and which extends substantially transversely with respect to a longitudinal direction of said shaft, and a substantially complementary opening arranged at said plate-shaped element, said plug being able to be fitted into said opening in a first position of rotation and, by rotating said plug to a second position of rotation, being secured against withdrawal from said opening.

15. The instrument system of claim 14, wherein said shaft has an inner shaft, on which said plug is formed, and an outer shaft which is axially displaceable but not rotatable with respect to said inner shaft, and which has, at a distal end, at least one projection which is offsystem by an angle with respect to said plug about said longitudinal direction and which, with said plug in said second position of rotation, engages in said opening.

16. The instrument system of claim 15, wherein said outer shaft is pretensioned in the distal direction by means of a spring force.

17. The instrument system of claim 1, wherein said connection mechamism is designed by said shaft having an inner shaft which has a fork at a distal end of said inner shaft, and by said plate-shaped element having at least two thread segments, and said fork can be coupled to said thread segments, in a cross-over arrangement secure against rotation, and by said shaft moreover having an outer shaft whose distal end can be screwed onto said thread segments.

18. The instrument system of claim 1, wherein said connection mechanism is designed by said shaft having an inner shaft and an outer shaft, said outer shaft being able to be screwed onto said plate-shaped element, and said inner shaft being able to be secured on said outer shaft by means of a securing element and being able to be withdrawn from said outer shaft by loosening of said securing element.

19. The instrument system of claim 1, wherein said plate-shaped element has at least one suction line with at least one suction opening.

20. The instrument system of claim 1, wherein said plate-shaped element has at least one irrigation line with at least one irrigation opening.

21. The instrument system of claim 1, wherein said shaft has at least one suction channel.

22. The instrument system of claim 1, wherein said shaft has at least one irrigation channel.

23. The instrument system of claim 18, wherein said securing element is at least one of a suction and irrigation attachment piece for attachment of at least one of a suction and irrigation line.

24. The instrument system of claim 1, wherein said plate-shaped element has at least one position sensor for detecting at least one of a position of said plate-shaped element and an area of connection of said plate-shaped element to said shaft.

25. The instrument system of claim 24, wherein said at least one position sensor is an electromagnetic sensor.

26. The instrument system of claim 1, wherein said plate-shaped element has at least one light-emitting element for radiating light through the cheek.

27. A Method for creating a surgical operation space by minimally invasive way in operations on the jaw, comprising
making a first incision through the cheek anterior to the ear, making a second incision in the mucous membrane adjacent to a site where said operation is to be carried out,
introducing a plate-shaped flat element through the mouth und through said second incision between the lower jaw bone and the cheek in the area of the jaw angle,
introducing an elongated shaft through said first incision towards said plate-shaped element,
connecting a distal end of said shaft with said plate-shaped element, and
drawing back said shaft after having being connected with said plate-shaped element to create said operating space between the lower jaw and a side of said plate-shaped element facing the lower jaw.

28. The method of claim 27, wherein said plate-shaped element is connected with said shaft such that said plate-shaped element extends at least approximately transversly with respect to said shaft.

29. The method of claim 27, wherein said plate-shaped element is connected with said shaft in an edge area of said plate-shaped element, said edge area being adjacent to the jaw joint when said plate-shaped element is inserted.

30. The method of claim 27, further comprising visual monitoring of said operation site by an endoscope through said shaft.

31. The method of claim 27, further comprising irrigating said operation site with an irrigation fluid through said shaft.

32. The method of claim 27, further comprising suctioning off tissue or liquids from said operation site through said plate-shaped element.

33. The method of claim 27, further comprising irrigating said operation site with an irrigation fluid through said plate-shaped element.

34. The method of claim 27, further comprising suctioning off tissue or liquids from said operation site through said shaft.
